(19) United States
(12) Patent Application Publication
(10) Pub. No.: US 2012/0203233 A1
(43) Pub. Date: Aug. 9, 2012

(54) CUSTOM SURGICAL CUTTING BLOCK DESIGN

(75) Inventors: Kenneth Kenji Yoshida, Costa Mesa, CA (US); Constantine Dumitrescu, San Francisco, CA (US)

(73) Assignee: ISM, INC., Hayward, CA (US)

(21) Appl. No.: 13/021,595

(22) Filed: Feb. 4, 2011

(51) Int. Cl. A61B 17/56; G06F 17/50

(52) U.S. Cl. 606/87; 703/1

(57) ABSTRACT

A method for making a surgical cutting guide and the guide made by this method. The method includes obtaining a number of cross sectional images of a patient joint taken at a defined interval. On each image a patient contour is imaged. These patient surface contours are used to generate a pattern of parallel ridges on a surgical cutting guide.

### CROSS-REFERENCE TO RELATED APPLICATION

This application claims priority from U.S. Provisional patent application Ser. No. ______, entitled “New Generation Advanced Orthopedic Cutting Guide”, filed Feb. 12, 2010.

### TECHNICAL FIELD

The disclosed embodiments relate to patient specific surgical templates, more specifically, its improved mating technology and manufacturing efficiency in surgical cutting guides.

### BACKGROUND

Baking For many, joint damage is unavoidable and debilitating damage may result from a number of causes such as repeated trauma, over-use, traumatic injuries and diseases (such as arthritis). In the physiological level, joint damage is caused when a flexible connective tissue (e.g. cartilage) which surrounds the bone and acts as a shock absorbing mechanism becomes eroded. Inevitably, bone to bone abrasion may occur but, even before then, cartilage degradation will result in pain and/or excessive joint play. Pain occurs due to joint inflammation caused by cartilage damage, joint wear and tear, and muscle strain and fatigue caused by stiff joints and limited motion.

For example, in the case of the knee joint, one way to restore joint movement and eliminate pain is a procedure called total knee replacement (TKR). In this procedure, a small portion of the femur distal region and tibia proximal region are removed using precise guides and instruments. The removed portions are replaced with metal and plastic prosthesis providing an artificial contact surface. Successful implants will restore the joint to a fully functional knee allowing normal leg mobility and eliminate pain.

Accurate implant alignment is important for joint replacement success. Excessive misalignment of implants to the extent of a few degrees may negatively affect the procedure outcome. Common adverse outcomes include tissue impingement, limited motion and pain. In some instances severe side effects may necessitate another operation. In order for a successful implant, an accurate and repeatable implant cutting block mating technology is required. Standardized surgical templates for clinical use offer only limited improvement in implant placement. Customized patient specific templates (patient matched surgical cutting guides) address the inherent limitations of standard templates or cutting guide instruments.

Currently, a series of MRI 2D images are used to construct a 3D solid model by means of interpolation. The 3D bone/cartilage models are then used to create a mating surface onto the cutting block. This bone/cartilage surface to cutting block surface (surface to surface mating technique) is used to assist in total knee replacement (TKR) surgical procedures in joint replacement in preparation for new prosthetic joints. With a typical MRI 2 mm spaced scanning, approximately 40 to 60 slices of the bone/cartilage around knee joint are required to determine the mating cutting block profile design.

For segmentation procedures, this approach requires total number of 80 to 120 slices to segment for both femur and tibia. With further irregularities in bone/cartilage profile, potentially due to excessive damage, 2 mm MRI scan may be too wide for a reliable cutting block due to the unpredictable nature of the arthritic damage ridden surfaces within the 2 mm gaps. There are inherent errors of deconstruction in surface to surface mating technique. These combinations of errors include MRI scan errors and cutting block software imaging errors of 2D segments to 3D construction due to interpolation between slices which reduce the chance for surgery success. For an accurate and repeatable cutting block design, these unknowns have to be eliminated or greatly reduced. Therefore, smaller segments (e.g. 1 mm) may be desired, doubling the number of scans. This method is costly, inefficient (doubling number of segmentation up to 160 to 240 slices) and only marginally better even at 1 mm segment resolution compared to 2 mm. Current MRI technology makes development of auto segmentation software difficult to determine bone/cartilage boundaries due to high MRI noise level. Consequently, a manual segmentation is the only option in this field and further adding to inefficiency. For these reasons 1 mm segmentation is not a good option for manufacturing.

What is needed is a new and improved method of custom cutting block with increased mating accuracy, resulting in a very effective and high manufacturing productivity. In order to achieve this, cutting blocks need to be designed directly from 2D segmentation data without interpolation between slices, resulting in bone/cartilage surface to cutting block line mating, or surface to line mating technique to be utilized.

### SUMMARY

In a first embodiment, a method for designing a patient conforming shape for a custom patient surgical cutting block includes an initial step of obtaining medical images taken at a known spacing interval. These images are input into a computer, which uses the images to determine a plurality of contour lines. Each contour line is derived from one image, although not ever image needs to be used. The computer then generates a patient conforming shape, which includes a plurality of spaced ridges that conform to the contour lines. The ridges are spaced apart at a spacing determined by the known spacing interval.

The ridges may have flat tips, pointed tips, rounded tips, or some combination of these types of tips. In some instances, the medical images will be generated from multiple slice directions. These images can generate contour lines that then can be used to make a surgical cutting guide with patient contour ridges that intersect. Such intersecting ridges may be either continuous or non continuous.

The above method may also be used to generate a novel surgical cutting block. This block may include a cutting slot, a plurality of pin holes, or a stabilizing hook.

### BRIEF DESCRIPTION OF THE DRAWINGS

FIGS. 1A-D illustrate the prior art method of surgical block design.

FIG. 1A is a side view of MRI imaging of a patient joint (in this case a knee).

FIG. 1B is a front perspective view of medical images.

FIG. 1C is a front view of a single image of FIG. 1B.

FIG. 1D is a side perspective view of 3-D imaging of bone end imaged in FIG. 1B.

FIG. 1E is the bone of FIG. 1D with adherent surgical cutting guide overlaying the bone.

FIG. 2A is a front view of a sagittal cross sectional image of a knee.

FIG. 2B is a front view of an axial cross sectional image of a knee.

FIG. 2C is a front view of a coronal cross sectional image of a knee.

FIG. 3A is a front view of a stack of images of a joint.

FIG. 3B is a bone profile assembled from the images of FIG. 3A.

FIG. 3C is a cross section of the bone shape of FIG. 3B.

FIG. 4A is a top perspective view of a ridge profile.

FIG. 4B is a cross sectional view of a cutting block making to a patient bone surface with lines indicating imaging sections.

FIG. 4C is a cross sectional detail of FIG. 4B.

FIGS. 5A-5C is a cross sectional view of custom cutting block design in which the contour ridges are spaced at t, 2t and 4t in the respective figures.

FIG. 6A shows a cross section of ridges having flat tips.

FIG. 6B shows a cross section of ridges having pointed tips.

FIG. 6C shows a cross section of ridges having rounded (radial) tips.

FIG. 6D shows a cross sections the three tips of FIGS. 6A-C on a rounded surface.

FIG. 7A shows a patient conforming side of a sagittal ridge design on a surgical cutting block.

FIG. 7B shows a patient conforming side of a coronal ridge design on a surgical cutting block.

FIG. 7C shows a patient conforming side of an axial ridge design on a surgical cutting block.

FIG. 7D shows a patient conforming side of a sagittal and coronal ridge design on a surgical cutting block.

FIG. 7E shows a patient conforming side of a sagittal and axial ridge design on a surgical cutting block.

FIG. 7F shows a top view of a surgical cutting block on a patient bone.

FIG. 8A shows a top perspective view of continuous intersecting ridges.

FIG. 8B shows a top perspective view of non-continuous intersecting ridges.

FIG. 9A is a front view of a distal femur with a pin set in the trochlear groove.

FIG. 9B is a front three dimensional perspective view of the distal femur with pin.

FIG. 10A is a side perspective view of a cutting block installation on distal femur including pin feature.

FIG. 10B is a side perspective view of a pin.

FIG. 10C is a cross sectional view of the view of FIG. 10A.

FIG. 11A is a top perspective view of the proximal tibia.

FIG. 11B is a side perspective view of the proximal tibia.

FIG. 11C is a top perspective view of the proximal tibia with a surgical cutting guide having a hook.

FIG. 11D is a view of the patient conforming surface of the guide shown in FIG. 11C.

FIG. 12A is a plan view of a guide manufacture.

FIG. 12B is a flow chart of the guide manufacture.

FIG. 13 is an expanded flow chart of the guide manufacture.

### DETAILED DESCRIPTION

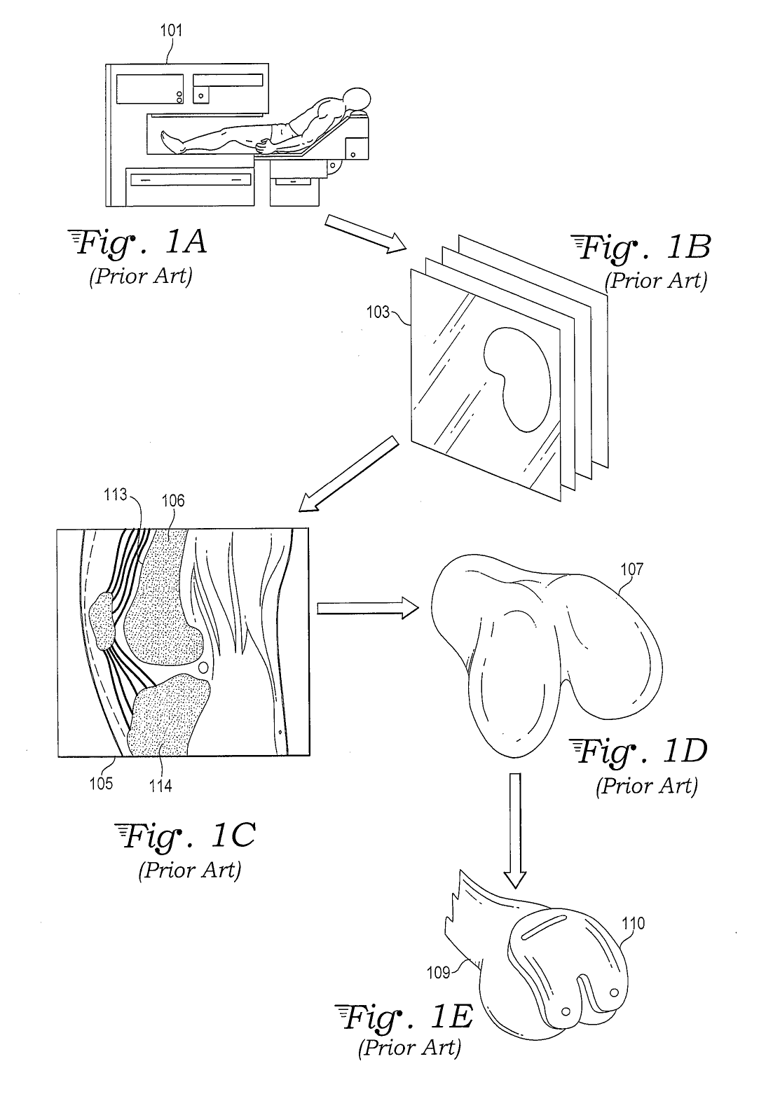

Disclosed herein is a system and method for manufacturing surgical cutting blocks such as those used in total knee replacement (TKR) cutting blocks having, improved mating accuracy. FIG. 1A-1E illustrates the workflow of current manufacturing of patient specific cutting guide 110. The first step shown in FIG. 1A is that the patient undergoes MRI scanning process using MRI 101 to image the area around the arthritis knee joints. During MRI scanning images are generated, such as image 103 of FIG. 1B. As shown in FIG. 1C, the patient's knee joint MRI 105 shows the distal femur 106 and proximal tibia 114 and ligament 113. The knee is scanned to provide the series of MRI slices, as shown by image 103 in FIG. 1B. These may be 2 mm slice thickness along the sagittal direction, resulting in the image set of FIG. 1B. After scanning, the sagittal scanning signals are digitized to provide the series of images in grayscale DICOM format. The series of the MRI slices are segmented manually for construction of 3D solid model of distal femur 107 shown in FIG. 1D. In the 3D constructed distal femur model 107, the current methods utilize the interpolation between MRI segmented slices. With the 3D femur model 107, the custom cutting block 110 is manufactured to be used in operating room during surgery. The custom cutting block 110 surface features obtained from 3D model 107 mates with patient's distal femur bone/cartilage surface 109 during surgery.

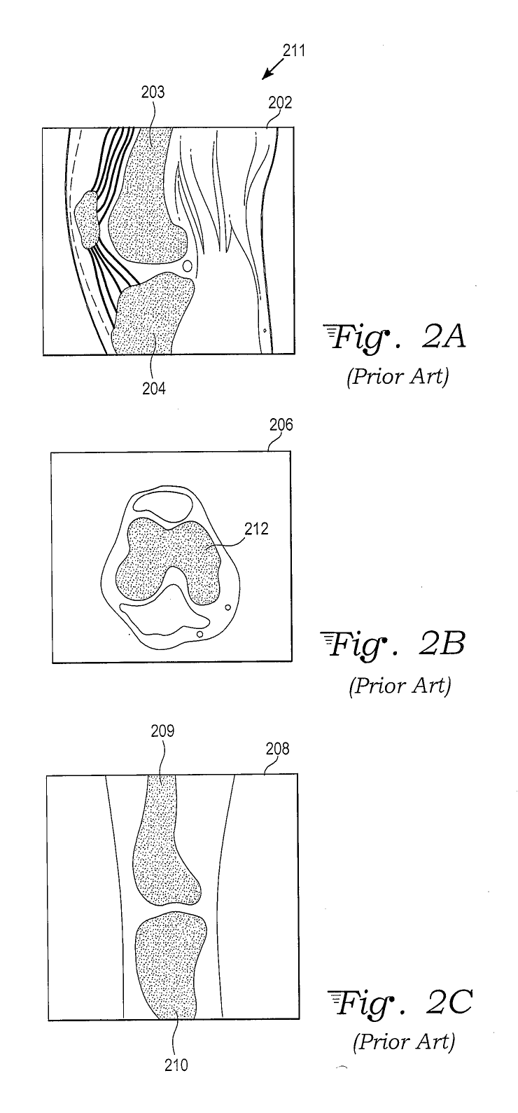

FIGS. 2A-2C illustrates a patient's 2D MRI images 211 in sagittal (2A), axial (2B) and coronal (2C) views. One of the sagittal images 202 contains the distal femur 203 and proximal tibia 204 in grayscale which assists in differentiating bone and any other soft tissues surrounding it. The series of the axial slice images also are captured with equal space or thickness as the knee joint is scanned from the femur down to the tibia. An axial slice 206 shows the distal femur 212 and clearly illustrates the trochlear groove profile. A series of coronal images are slices captured as the imaging moves from anterior to the posterior sections of the knee. A coronal slice 208 shows the distal femur 209 and proximal tibia 210. These sliced images can be used in manual segmentation of the bone/cartilage profile at the knee and is converted into 3D solid model 107. The process of 3D solid model construction entails using the 2D segmentation data and interpolating between slices via linear or polynomial interpolation which adds inaccuracies between slices. Furthermore, the most accurate bone/cartilage profile information obtained from original manual segmentation can be lost during smoothing and decimation process in 3D construction. Especially, the segmentation dealing with excessive bone/cartilage wear, the interpolation between slices becomes unpredictable although the accurate 2D manual segmentation on each MRI slice is performed. As a result, the current 2D to 3D construction method can decrease the mating accuracy.

In general, the accumulated errors in joint replacement cutting block designs are as follows:

[symbol]

Total= [symbol] MRI Image+ [symbol] 3D Construction (1)

[symbol]

MRI Image= [symbol] MRI Noise+ [symbol] MRI Volume Averaging. (2)

[symbol]

3D Construction= [symbol] connectivity+ [symbol] Smoothing function+ [symbol] decimation (3)

where, [symbol]
connectivity is an error caused by linear or polynomial connection between slices, [symbol] Smoothing function represents the graphic meshing smoothing error, and [symbol] decimation represents a graphic function error in minimizing mesh count in describing the 3D model. [symbol] MRI Noise and [symbol] MRI Volume Averaging are errors related to current MRI technology. It is important to note that [symbol] MRI Image< [symbol] 3D Construction. As a result, the improvement of the custom cutting block clearly depends on minimizing or eliminating the error in 3D construction.

MRI image error in Equation (2) within the MRI machines cannot be improved without drastic advancements in its technology. On the other hand, 3D construction error, which is the primary source for cutting block mating inaccuracy in Equations (3), can be minimized or removed by designing its contact at the bone/cartilage contours of the known lines generated by scans providing a reliable fit. Thus, Equation (1) becomes

[symbol]

Total= [symbol] _{MRI} Image.

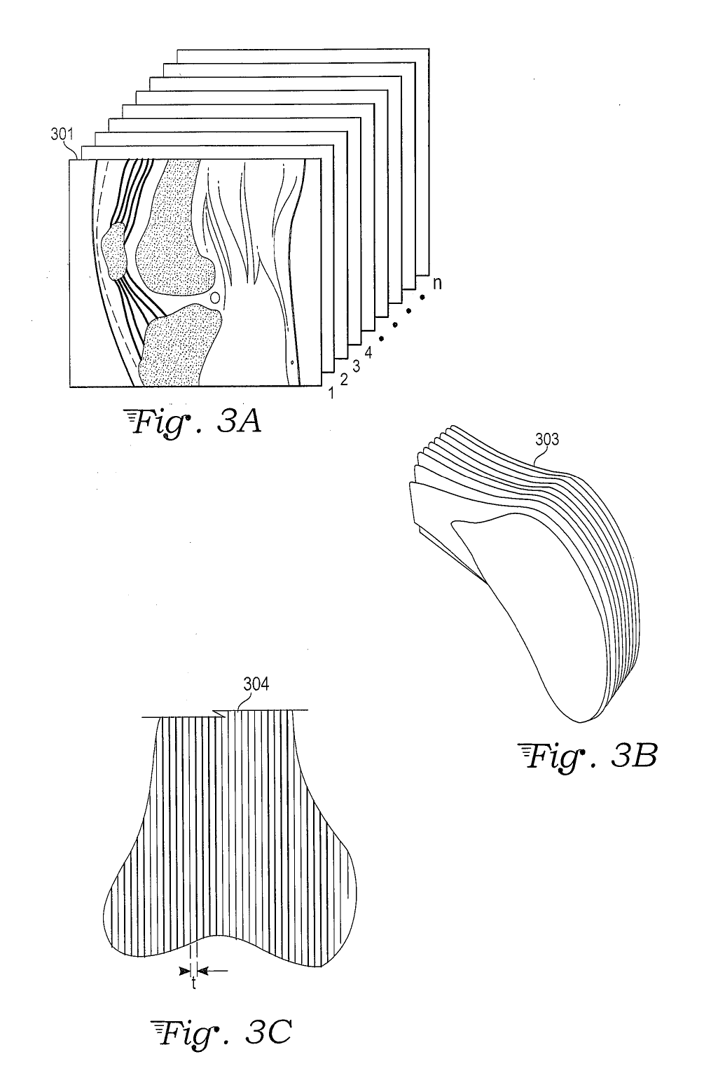

FIGS. 3A-3C represents the basis for the new mating technique; three dimensional construction in surface to line mating technology. MRI slice images 301, equally spaced into n number of slices, are manually segmented as per current technology. But unlike the current method of design and manufacture of surgical cutting blocks, instead of using the segmented information and interpolating between slices followed by applying a smoothing function, the raw manual segmentation data is used to position each slice exactly as taken from 2D images but in three dimensional space 303 as shown in FIG. 3B. The profiles generated by these lines 304 shown in FIG. 3C in coronal view demonstrates the bone/cartilage of knee joint with omitted information or without interpolation between slices at a given thickness t. The missing information between slices is not crucial for an accurate mating system since this data will not be used in designing and manufacturing the custom cutting block. The custom cutting block merely needs to be designed to make contact at the generated 2D segmented line curves 303 and not via surfaces. In addition, since [symbol] MRI Image< [symbol] 3D Construction, the largest portion of the error has been eliminated allowing for a more precise mating technique.

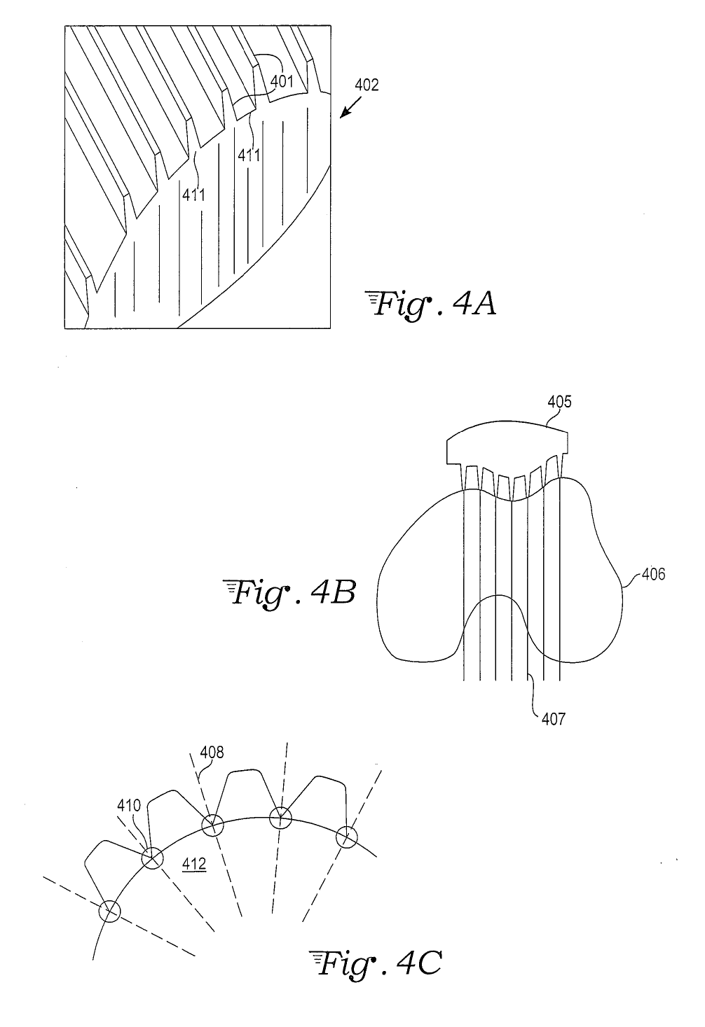

FIGS. 4A-4C shows details of the ridges on a surgical cutting block. In FIG. 4A, the custom cutting block 402 shows the series of basic features of peak 401-valley 411-peak 401. The peak features 401 represent the segmented lines of MRI slices which eventually mate on the patient's anatomical surface in the operating room. The 3D line profile of the bone/cartilage information, generated directly from raw 2D segmentation, is used to design the mating custom cutting block. The axial section view of FIG. 4B illustrates the custom cutting block 405 mated to the patient's distal femur 406 based on the sagittal MRI slices (represented by lines 407). It is noted from the detailed axial section view of FIG. 4C, the contacts between the custom cutting block 408 and distal femur 412 are made and shown as points 410. The custom cutting block 408 shows peaks (401 in FIG. 4A) as lines which contact the bone/cartilage, and the valleys (411 in FIG. 4A) are the areas between the slices of bone/cartilage without interpolation and used not to make contact between slices. The fundamental idea behind the design is to use the raw information “as is” where possible and eliminate inexact information thereby making this contact method much more accurate.

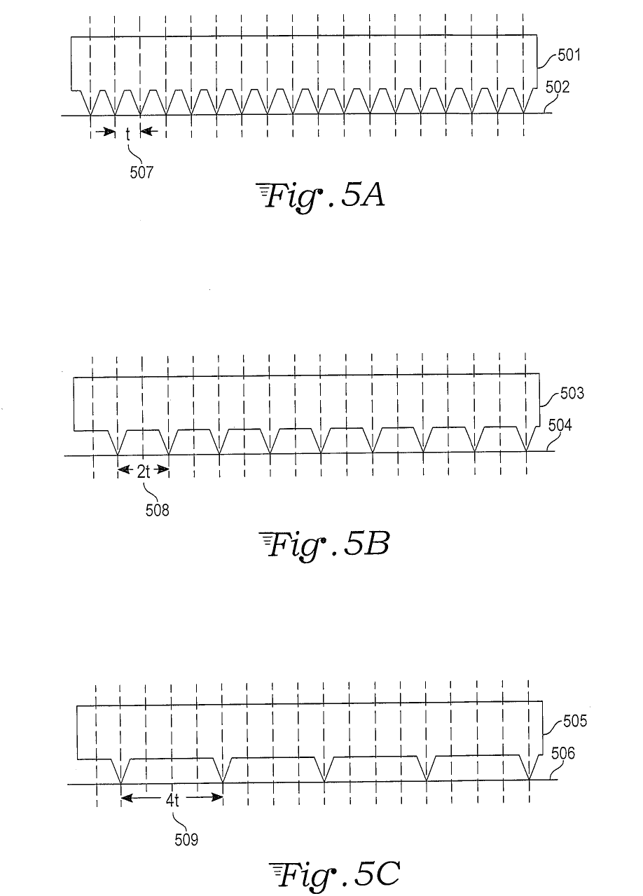

FIGS. 5A-5C represents the custom cutting block with an increase of efficiency and productivity by requiring fewer segmentations. A typical custom cutting block of the line contact design 501 with MRI slice thickness t mates on the bone/cartilage surface 502. Moreover, one of the important advantages of the custom cutting block is that the total number of segmentations can be reduced by half (2t of FIG. 5B) or one-quarter (4t of FIG. 5C), and still maintain its accuracy. The custom cutting block 503 with the line contacts, 2t (508) mates on the surface 504. Furthermore, the custom cutting block 505 with the line contacts, 4t (509), mates on the surface 506. The surface characteristics of the distal femur and proximal tibia allow fewer manual segmentations without a loss of mating accuracy regardless of excessive bone/cartilage damage. This can significantly reduce the amount of work required for making a surgical cutting block. Currently, a physician must review images and determine if the bone contour for the shape of the surgical cutting guide. With the present invention, removal of some slices can reduce this work by a half or three quarters. In addition, a user can select images that are to be used. For the rounded ends of a conforming patient bone surface, one or two contoured ridges on each side of a rounded feature should be sufficient to place the surgical cutting jig in an accurate position. FIGS. 5B and 5C show regular spacing, with every third image utilized. However it is possible to use an irregular pattern (e.g. images 1, 3, 7, 9, 12, etc.). This allows image selection, in which the poorest images could be excluded from use in design of the surgical cutting guide.

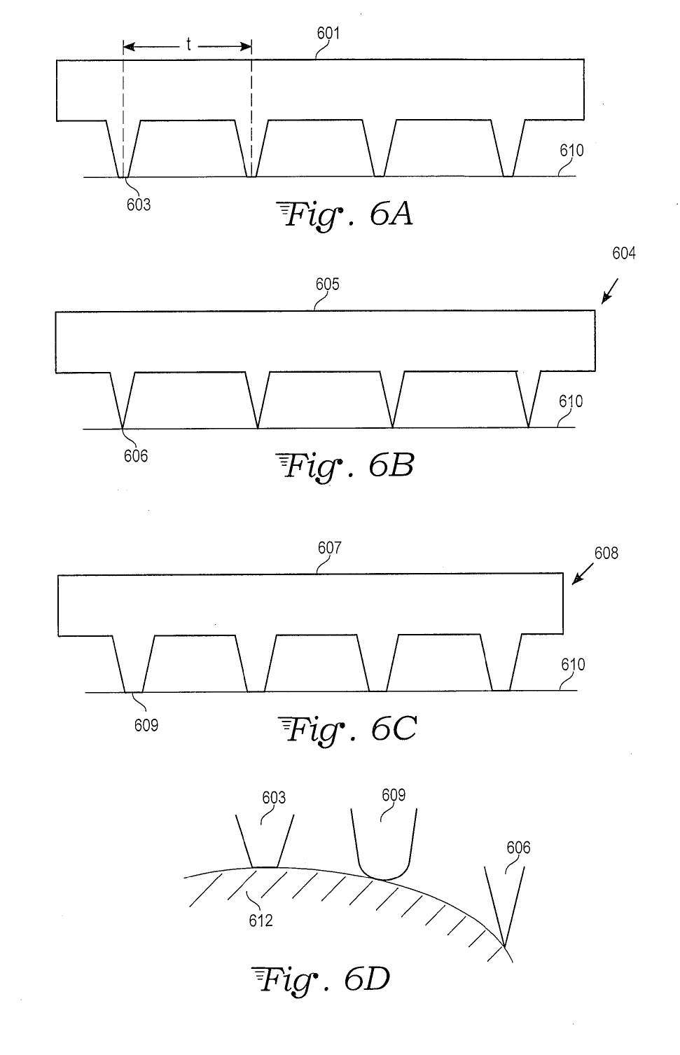

FIGS. 6A-6D shows several contact tip features of the custom cutting block contact lines with t spaced segmentation. In FIG. 6A, the custom cutting block 601 includes the flat tip 603 of the contour ridge surface feature with around 1 mm width to mate on the bone/cartilage surface 610. In FIG. 6B, the custom cutting block 605 includes the sharp tip 606 feature that lines up with the segmented bone/cartilage surface 610. In FIG. 6C, the custom cutting block 607 has the rounded tip feature 609 with around 1 mm diameter to make line contact on the bone/cartilage surface 610. The combination of the three tip designs can be used as shown in FIG. 6D. The flat and radial feature 603, 609 can be useful where bone/cartilage surface 612 is reasonably flat; whereas the sharp tip, feature 606 can be used on non-flat surface, especially, bone/cartilage damaged area, to add additional mating stability.

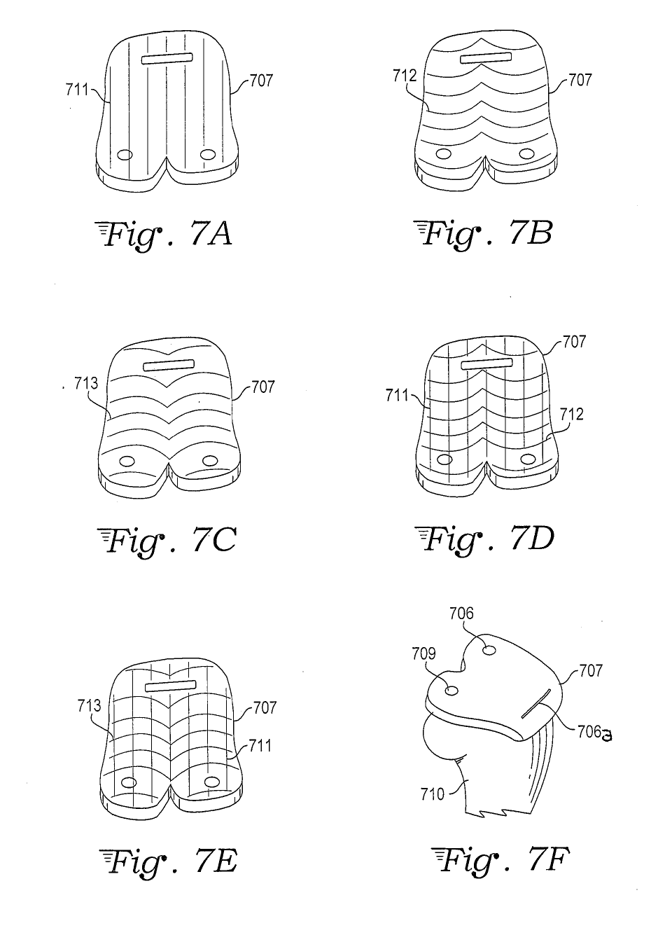

FIGS. 7A-7F represents the custom cutting block 707 designed with the line contact directions of sagittal, coronal, axial MRI images. With reference to FIG. 7F the custom cutting block 707 including the hole features 706, 709 and cutting slot feature 706a. The shape conforms with mates the patient's distal femur 710. Based on sagittal direction MRI, FIG. 7A shows the custom cutting block 707 having the contact peaks 711 running from the top to bottom of the surgical guide. In the coronal MRI and axial MRI, the contact peaks both run in the left to right direction (Seen in FIGS. 7B and 7C as ridges 712, 713 respectively). The ridge contours are determined by the differences in slice directions. In the sagittal/coronal surgical guide the patterns of ridges extend in the both top to bottom 711 and left to right 712 (FIG. 7D). In the sagittal/axial surgical guide the patterns of ridges extend in the both top to bottom 711 and left to right 712 (FIG. 7E). In these figures, the resulting form is a mesh-like intersecting ridge patterns onto the surface of the custom cutting block 707. Any combination of MRI views except for coronal and axial combination can be used to design the custom cutting block; furthermore, the mating accuracy can be increased.

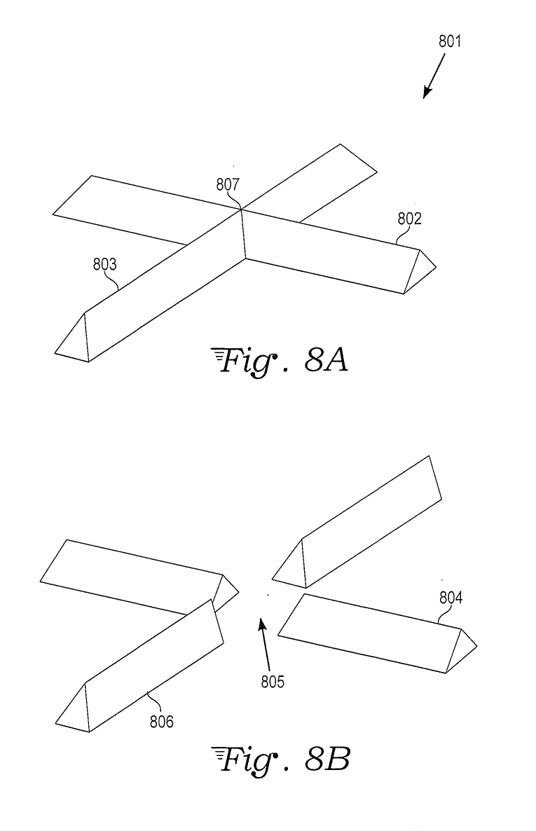

FIG. 8A represents the custom cutting block contact line intersections 801, where the ridges 802, 803 are formed by segmenting the combinations of MRI scan images (sagittal+coronal or sagittal+axial). The intersections of the lines 802, 803 can be continuous, as shown in FIG. 8A. Alternatively, as shown in FIG. 8B, intersecting ridges can be designed with a void 805 where ridges 804, 806 meet. The continuous intersection provides a conforming fit to a patient bone surface where there is no or slight bone/cartilage wear. In contrast, for damaged area of the bone/cartilage arthritis, the void feature 805 at those intersections can be useful since the bone/cartilage damaged area usually contain the imaging error.

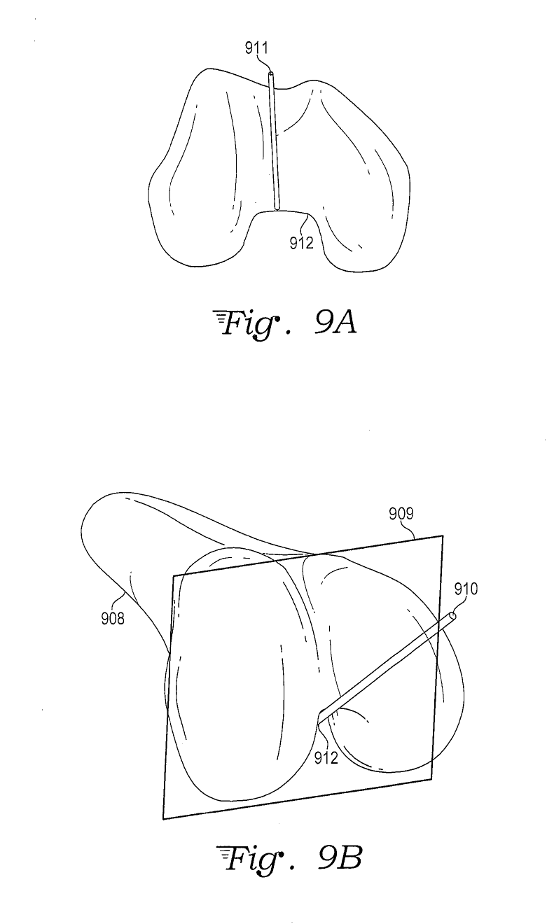

FIGS. 9A and 9B represents the axial view of the distal femur of a patient. The MRI would have scanned near the center of the distal femur to display the sagittal image around the trochlear groove notch 912 shown in FIG. 9A. The pin 911 would be inserted at this location to retain the guide. From the sagittal view 9B of distal femur 908, the lowest point of the trochlear groove 912 is identified with respect to the cut plain 909. With the angle of 40 to 50 degrees with respect to the cut plane 909, a stabilizing pin 910 is positioned near the notch 912 below the trochlear groove. The stabilizing pin 910 is designed with 3 to 5 mm diameter and provides the initial positioning on the custom cutting block to bone/cartilage surface. Once the stabilizing pin makes contact on the notch area 912, then the final positioning of the custom cutting block is performed in conjunction with the custom cutting block lines mating onto the patient's distal femur surface. The stabilizing pin also provides additional stability; consequently, the mating of custom cutting block on the distal femur surface becomes highly reliable.

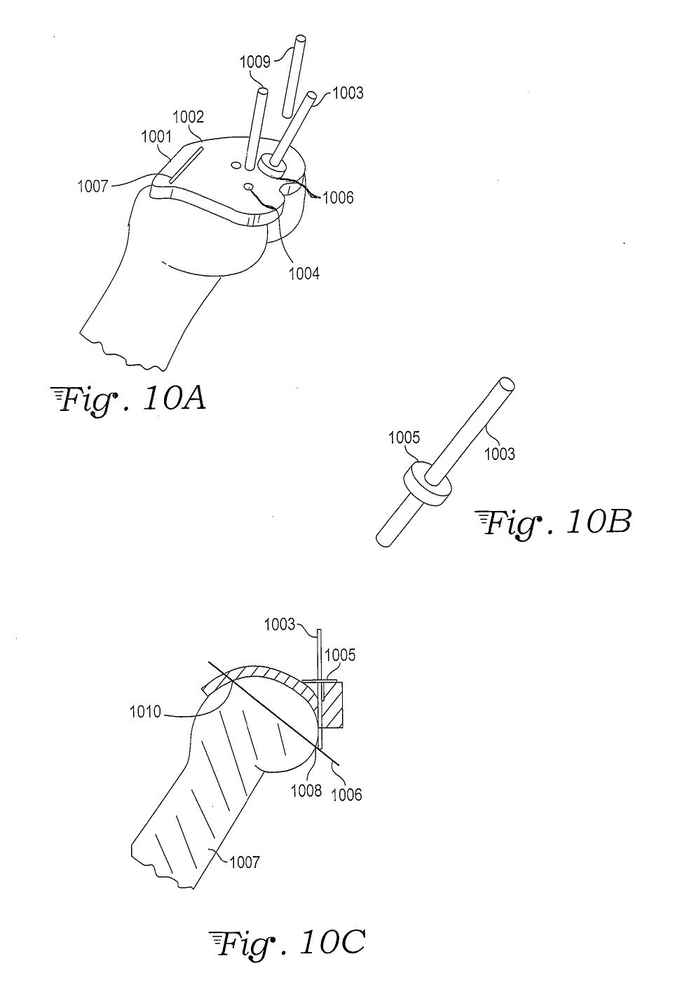

FIG. 10A shows the custom cutting block 1002 mating on the distal femur surface 1001 with the attachable/detachable stabilizing pin 1003 inserted into the hole 1006. As shown in FIG. 10B the stabilizing pin 1003 has an annular lip 1005. As seen in FIG. 10C, the annular lip is retained at the notch 1008 below the trochlear groove 1010 when the custom cutting block is pushed up for initial position. Again with reference to FIG. 10A, drilling holes 1004 are provided to install pins 1009 on the custom cutting block 1002 for the final positioning. The stabilizing pin is removed 1003 so that it allow clearance for the cutting tool 1006 when inserted into cutting slot 1007 to remove bone during TKR operation.

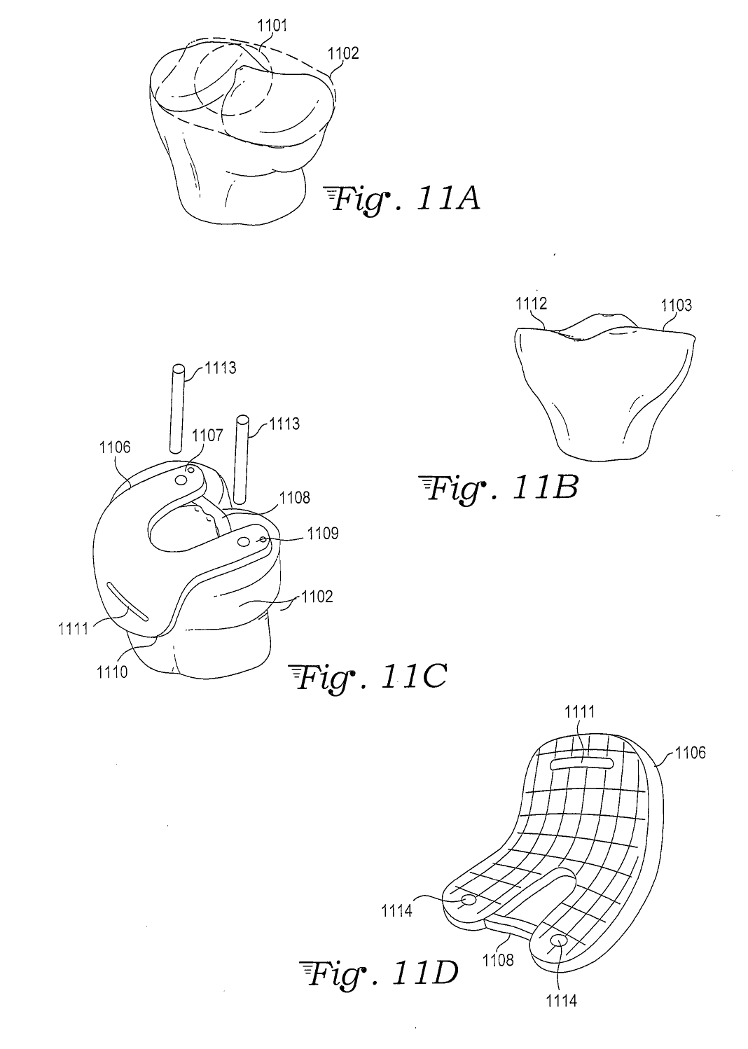

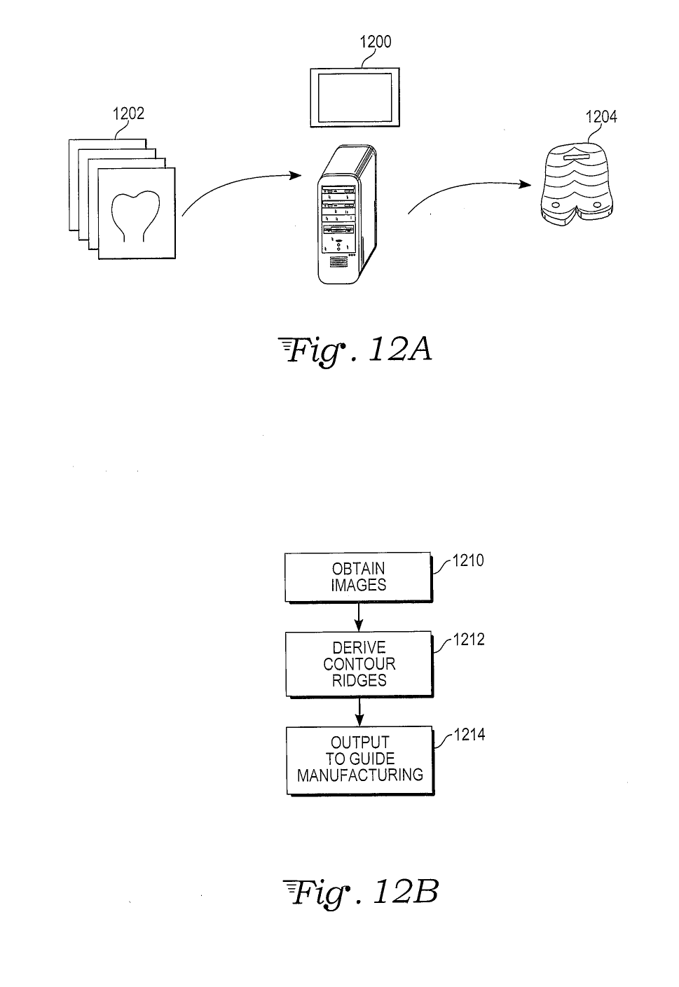

FIG. 11D shows the custom cutting block 1106 with the cutting slot 1111 and drilling holes 1114. A patient conforming surfaces has a net work of ridges that allow mating on the top surface of the tibial plateau and tibial anterior surface on the proximal tibia. As seen in FIG. 11A, in the middle of the tibial plateau 1102 is the tibial spine 1101. FIG. 12B shows a peak 1112 on plateau 1103. As seen in FIG. 11C, with the series of the points extending from lateral plateau 1107 to medial plateau 1109, the tibial custom cutting block is designed to include a stabilizing hook 1108 feature at the posterior spine position. The custom cutting block 1106 of FIG. 11D is shown with mesh-line shape line contact features includes the hook feature 1108. Once hooking onto the tibial spine with drilling hole pins, the custom cutting block positioning becomes reliable and ready for the cutting tool at the cutting slot 1111. Pins 1113 may be inserted to further retain the bone in place.

With reference to FIG. 12A, a computer 1200 receives a number of cross sectional medical images 1202 of a patient joint. Each image is captured at a known interval. The computer, either using automated processes or with user input, determines a bone contour line. This line is then used to manufacture a surgical cutting guide 1204 having ridges with a patient conforming shaped derived from the bone contour line.

The steps of this method are shown in FIG. 12B. First, in step 1210, the cross sectional images at a known spacing are obtained from a patient. Second, the computer derives the patient contour surfaces in step 1212. Finally, these patient conforming ridges are output to the manufacturing system that produces the surgical cutting block.

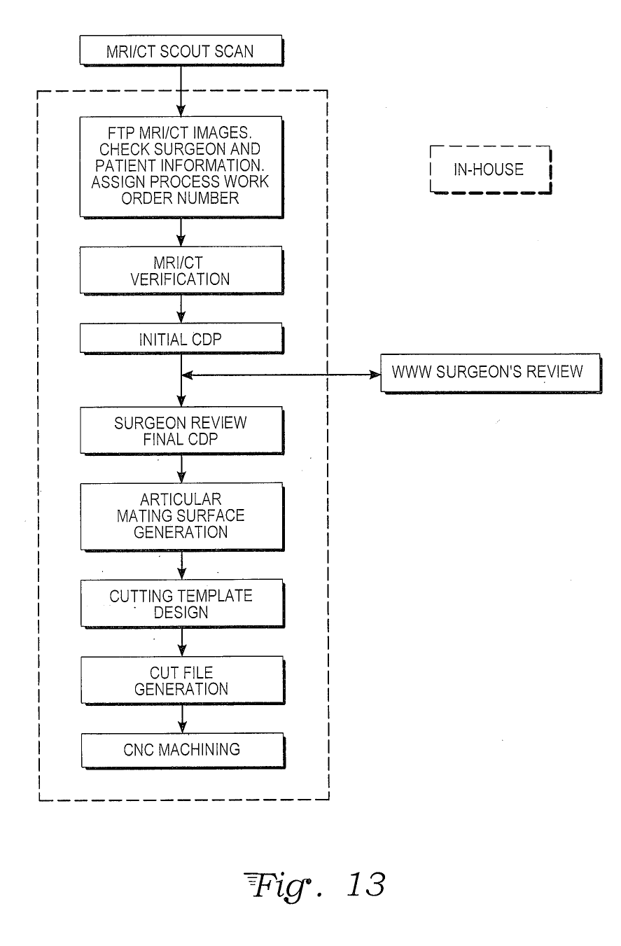

With respect to FIG. 13 a more detailed work plan is shown. The first step is the MRI/CT scan to generate the patient images at determined intervals. The second step is the assigning of the Surgeon and Patient information and process work order number to these images. The MRI/CT images verified. A surgeon may review an initial CDP to generate the final CDP. The computer will next generate an articular mating surface, make a template design, and generate a cut file. This is then used to machine the final surgical guide.

In the present illustrations, a knee replacement has been used as the example of the present method and devices. However it should be appreciated that the disclosed method and devices may be used for heel, shoulder, elbow, hip or other joint replacement. In addition, the prior art interpolation method of surgical cutting block manufacture requires constructing a surface that is designed to contact an entire surface onto which the block is place. In contrast, the present surgical block is designed to contact a much more limited area with a higher fidelity of contact shape. Not every slice of an image needs to be used. As explained with FIG. 5, a pattern of regular or irregular images may be used. This would allow the clearest images to be used, and ridges to be used that have the best fit onto the patient surface. In addition, it simplifies manufacturing of the surgical guide.

What is claimed is:

1. A method of determining a patient conforming shape for a custom surgical cutting block, the method comprising:
a) providing to a computer a plurality of medical images of a patient, said medical images taken at a defined spacing interval;
b) obtaining, using said computer, a plurality of contour lines each derived from one of said plurality of medical images; and
c) generating, using said computer, a patient conforming shape including a plurality of spaced ridges shaped to conform to said contour lines and spaced at said defined spacing interval.

2. The method of claim 1, wherein said plurality of spaced ridges includes a plurality of spaced ridges having a flat tip.

3. The method of claim 1, wherein said plurality of spaced ridges includes a plurality of spaced ridges having a rounded tip.

4. The method of claim 1, wherein said plurality of spaced ridges includes a plurality of spaced ridges having a pointed tip.

5. The method of claim 1, wherein said plurality of spaced ridges includes at least two of spaced ridges having a flat tip, spaced ridges having a rounded tip, and spaced ridges having a pointed tip.

6. The method of claim 1, wherein step a) includes providing to a computer a first set of medical images of a patient taken at a first slice direction, said medical images taken at a first defined spacing interval and providing a computer a second set of medical images of a patient taken at a second slice direction;
step b) includes obtaining, using said computer, a first plurality of contour lines each derived from one of said first set of medical images and a second plurality of contour lines each derived from one of said second set of medical images; and
step c) includes generating, using said computer, a patient conforming shape including a plurality of intersecting ridges, said ridges including a first plurality of ridges conforming to said first plurality of contour lines and spaced at a said first defined spacing interval and a second plurality of ridges conforming to said second plurality of contour lines and spaced at said second defined spacing interval.

7. The method of claim 6, wherein said intersecting ridges are continuous.

8. The method of claim 6, wherein said intersecting ridges are not continuous.

9. A patient specific surgical cutting block, comprising:
a mating surface on said surgical block configured to matingly fit with a patient surface, the mating surface created by steps including:
a) providing to a computer a plurality of medical images of a patient, said medical images taken at a defined spacing interval;
b) obtaining, using said computer, a plurality of contour lines each derived from one of said plurality of medical images; and
c) generating, using said computer, a patient conforming shape including a plurality of spaced ridges shaped to conform to said contour lines and spaced at said defined spacing interval.

10. The cutting block of claim 9, wherein said plurality of spaced ridges includes a plurality of spaced ridges having a flat tip.

11. The cutting block of claim 9, wherein said plurality of spaced ridges includes a plurality of spaced ridges having a rounded tip.

12. The cutting block of claim 9, wherein said plurality of spaced ridges includes a plurality of spaced ridges having a pointed tip.

13. The cutting block of claim 9, wherein said cutting block includes a cutting slot.

14. The cutting block of claim 9, wherein said cutting block includes a plurality of pin holes.

15. The cutting block of claim 9, wherein said cutting block includes a stabilizing hook.

16. The cutting block of claim 9, wherein said spaced ridges include a plurality of intersecting spaced ridges.

17. The cutting block of claim 16, wherein said intersecting spaced ridges are continuous.

18. The cutting block of claim 16, wherein said intersecting spaced ridges are non continuous.
